(19) United States
(12) Patent Application Publication
(10) Pub. No.: US 2021/0145816 A1
(43) Pub. Date: May 20, 2021

(54) PHARMACEUTICAL FORMULATION OF LONAFARNIB WITH A SULFOBUTYLETHER BETA-CYCLODEXTRIN

(71) Applicant: Cyclolab Cyclodextrin Research and Development Laboratory Ltd., Budapest (HU)

(72) Inventors: István PUSKÁS, Budapest (HU); Lajos SZENTE, Budapest (HU); Tamas SOHAJDA, Halasztelek (HU)

(73) Assignee: Cyclolab Cyclodextrin Research and Development Laboratory Ltd., Budapest (HU)

(21) Appl. No.: 16/685,632

(22) Filed: Nov. 15, 2019

(51) Int. Cl. A61K 31/4545; A61K 47/40

(52) U.S. Cl. CPC A61K 31/4545; A61K 47/40

(57) ABSTRACT

Using sulfobutylether β-cyclodextrin a novel, physically transformed, molecularly dispersed forms lonafarnib may be prepared having pharmaceutical utility.

### BACKGROUND OF THE INVENTION

This invention relates to novel physically transformed, molecularly dispersed forms of pharmaceutical utility comprising lonafarnib and sulfobutylether β-cyclodextrin.

### FIELD OF THE INVENTION

Lonafarnib is a farnesyltransferase inhibitor (FTI), a synthetic tricyclic halogenated carboxamide with antineoplastic properties. Lonafarnib has been investigated in a human clinical trial as a treatment for progeria, which is an extremely rare genetic disorder in which symptoms resembling aspects of aging are manifested at a very early age. For those with progeria, it has been shown that the drug reduces the prevalence of stroke and transient ischemic attack, and the prevalence and frequency of headaches while taking the medication. A phase II clinical trial was completed in 2012, which showed that a cocktail of drugs that included lonafarnib and two other drugs met clinical efficacy endpoints that improved the height and diminished the rigidity of the bones of progeria patients. Lonafarnib has a low aqueous solubility (0.0008 mg/mL), thus, development of an aqueous intravenous or oral formulation is difficult.

### DESCRIPTION OF THE RELATED ART

A prior publication describing formulation of lonafarnib into a liquid vehicle with a cyclodextrin is known (Hernandez et al., Science Translational Medicine, 11, eaat3005 (2019)), but based on the disclosed data, even using high concentration (20%) of 2-hydroxypropyl beta cyclodextrin (hydroxypropyl betadex), a suspension of lonafarnib was obtained for the (pre)clinically relevant concentration of the drug (12 mg/ml). The experiment was reproduced by Cyclolab Ltd., and indeed, only rough suspension, unsuitable for parenteral application was obtained. Consequently, present invention provides a significant formulation improvement over the teachings of said journal publication.

International Patent Application WO 91/11172 discloses sulfoalkylether cyclodextrin derivatives including sulfobutylether β-cyclodextrin. Preparation of sulfobutylether β-cyclodextrin used for the present invention is disclosed in Hungarian Patent HU228817.

### BRIEF SUMMARY OF THE INVENTION

Using sulfobutylether β-cyclodextrin a novel, physically transformed, molecularly dispersed forms lonafarnib may be prepared having pharmaceutical utility.

### BRIEF DESCRIPTION OF THE OF THE DRAWING

FIG. 1 depicts the chromatogram of lonafarnib in the novel formulation as obtained by high pressure liquid chromatography (HPLC). Dimethyl sulfoxide (DMSO) is present in the chromatogram only for technical reasons (utilized in sample preparation)

### DETAILED DESCRIPTION OF THE INVENTION

According to the present invention, there is provided a pharmaceutical formulation comprising lonafarnib, or a pharmaceutically acceptable derivative thereof, and sulfobutylether β-cyclodextrin the average degree of substitution (DS) is in the range 5.9-6.6 as determined by nuclear magnetic resonance spectrometry (European Pharmacopoeia 2.2.33). This enhances molecular encapsulation resulting in enhanced lonafarnib solubility. This effect would not be anticipated because increasing the degree of substitution increases steric hindrance around the cavity of the cyclodextrin and would be expected to reduce complexation efficiency.

It is preferred in sulfobutylether β-cyclodextrin each O(CH₂)₄SO₃H present is in the form of an alkali metal salt (such as the sodium salt). This enhances the affinity of the molecule for lonafarnib, which is unexpected because lonafarnib is not charged.

Preferably, the formulation is for parenteral administration, for example, i.v. administration.

Generally, in aqueous intravenous and intramuscular formulations according to the invention, the lonafarnib will be present at a concentration of from 5 mg/ml to 50 mg/ml, for example 10 mg/ml to 30 mg/ml. The sulfobutylether β-cyclodextrin will be present in a molar ratio of lonafarnib:cyclodextrin derivative of from 1:1 to 1:10, for example 1:2 to 1:7, in particular 1:2 to 1:3. The formulations may be lyophilised (freeze dried) for storage prior to use, and made up with water when required.

In the following example, the sulfobutylether β-cyclodextrin has an average sulfobutylether substitution of 6.5 per cyclodextrin molecule, and each sulfobutylether unit is present as its sodium salt.

### Example 1

i.v. formulation of lonafarnib I. (liquid)

Lonafarnib (Sigma) 10.0 mg

Sulfobutylether β-cyclodextrin (Cyclolab Dexolve) of DS 5.9 160.0 mg

Water for injections (Ph. Eur).Total to 1.00 ml

Method:

1. With constant stirring, add the sulfobutylether β-cyclodextrin (SBECD) to 80% of the final volume of water for injections, and continue to stir until all the SBECD has dissolved.

2. Add the lonafarnib and dissolve with stirring.

3. Make the solution up to volume with water for injections.

4. Set pH to 3.5±0.5

5. Filter the resulting solution through a sterile 0.2 micrometer pore size polyethylene sulfone filter into a sterile container.

6. Fill 1 ml volumes into sterile vials, stopper and crimp.

### Example 2

i.v. formulation of lonafarnib II. (lyophilizate)

Lyophilize i.v. formulation of lonafarnib I. (liquid)

### Example 3

i.v. formulation of lonafarnib III.

Lonafarnib (Sigma) 10.0 mg

Sulfobutylether β-cyclodextrin (Cyclolab Dexolve) of DS 6.6 160.0 mg

Water for injections (Ph. Eur).Total to 1.00 ml

Method:

1. With constant stirring, add the sulfobutylether β-cyclodextrin (SBECD) to 80% of the final volume of water for injections, and continue to stir until all the SBECD has dissolved.

2. Add the lonafarnib and dissolve with stirring.

3. Make the solution up to volume with water for injections.

4. Set pH to 3.5±0.5

5. Filter the resulting solution through a sterile 0.2 micrometer pore size polyethylene sulfone filter into a sterile container.

6. Fill 1 ml volumes into sterile freeze drying or injection liquid vials, stopper and crimp.

7. Lyophilise (optional).

### Example 4

i.v. formulation of lonafarnib IV.

Lonafarnib (Sigma) 12.0 mg

Sulfobutylether β-cyclodextrin (Cyclolab Dexolve) of DS 6.4 200.0 mg

Water for injections (Ph. Eur), Total to 1.00 ml

Method:

1. With constant stirring, add the sulfobutylether β-cyclodextrin (SBECD) to 80% of the final volume of water for injections, and continue to stir until all the SBECD has dissolved.

2. Add the lonafarnib and dissolve with stirring.

3. Make the solution up to volume with water for injections.

4. Set pH to 3.5±0.5

5. Filter the resulting solution through a sterile 0.2 micrometer pore size polyethylene sulfone filter into a sterile container.

6. Fill 1 ml volumes into sterile freeze drying vials injection liquid, stopper and crimp.

7. Lyophilise (optional).

### Example 5

HPLC analysis of lonafarnib i.v. formulations

HPLC method:

Column: Kinetex C18

Eluene: A-channel: purified water+0.05% formic acid
B-channel: acetonitrile+0.05% formic acid

Gradient:

| | | |
|---|---|---|
| 0 min | 50% | 0.8 |
| | | ml/minute |
| 10 min | 80% | 0.8 |
| | | ml/minute |

Injection.: 2 microlitres

Column temperature: 25° C.

Diluent: 50% acetonitrile+0.05% formic acid

Detection: DAD

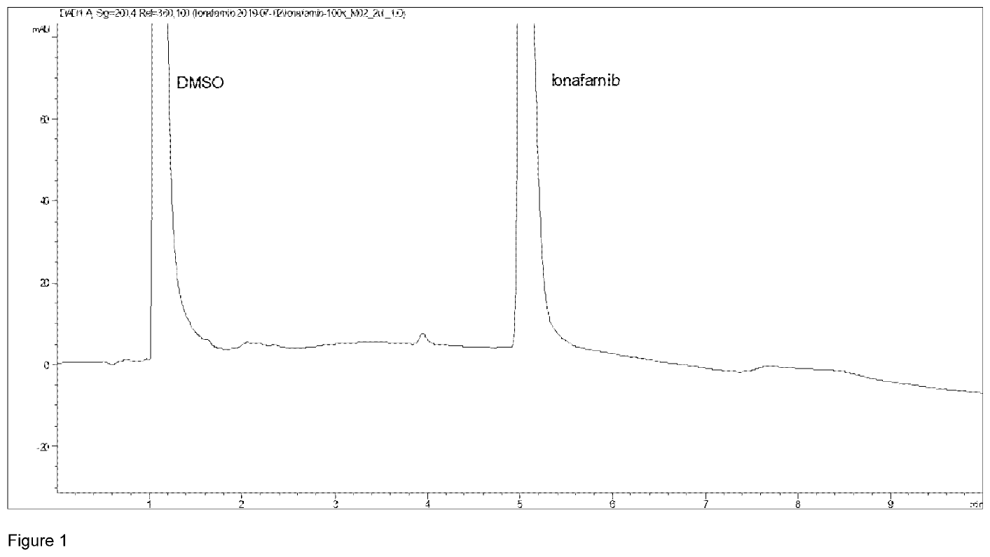

The HPLC chromatogram of the obtained liquid is shown in FIG. 1. The analysis has shown that the purity of the dissolved substance is 99.95±0.05% based on Area %.

What is claimed is:

1. A pharmaceutical formulation comprising lonafarnib and sulfobutylether β-cyclodextrin.

2. A formulation as claimed in claim 1, wherein the average number of O(CH₂)₄SO₃H groups per molecule of sulfobutylether β-cyclodextrin is in the range 5.9-6.6.

3. A formulation as claimed in claim 1 wherein each O(CH₂)₄SO₃H present is in the form of an alkali metal salt.

4. A formulation as claimed in claim 1, which is adapted for parenteral administration.

5. A solution made by making up a lyophilized formulation, as claimed in claim 1, in water.
